(19) United States
(12) Patent Application Publication
(10) Pub. No.: US 2017/0319375 A1
(43) Pub. Date: Nov. 9, 2017

(54) Methods of Improving Hygiene, and Articles and Kits for Use in the Same

(71) Applicant: Nettforsk AS, Arendal (NO)

(72) Inventors: Baard Johannessen, Arendal (NO); Inga Kaasen, Arendal (NO)

(21) Appl. No.: 15/526,136

(22) PCT Filed: Nov. 11, 2015
(86) PCT No.: PCT/EP2015/076305

§ 371 Date: May 11, 2017

(30) Foreign Application Priority Data
Nov. 11, 2014 (GB) 1420065.3
Jan. 20, 2015 (GB) 1500899.8

(51) Int. Cl. A61F 5/50; A41D 31/00; A41D 19/015

(52) U.S. Cl. CPC A61F 5/50; A41D 19/015; A41D 31/0077

(57) ABSTRACT

The invention relates to methods of improving hygiene in human subjects by discouraging contact between the hands and face. The method uses a hand covering article such as a glove which is typically coated in an irritant such as chili, whereby when the face is touched an unpleasant sensation occurs. The hand covering article is typically worn for prolonged periods to train the subject to avoid subconsciously touching the face. Kits containing the hand covering articles as well as compositions containing irritants for forming the articles are also disclosed.

### FIELD OF THE INVENTION

The present invention relates to a method of improving hygiene of a human subject by discouraging contact between the hands and the face. In particular, the method of the present invention discourages contact between the hands and the mucosal membranes of the face (eyes, mouth and nose). The present invention further relates to a method of changing the behaviour of a human subject.

### BACKGROUND

The skin provides an impermeable barrier to pathogens which could otherwise invade the body and cause illness. Such pathogens can however enter the body via open wounds, by ingesting contaminated foods and by inhaling airborne pathogens.

Another way in which pathogens can enter the body is via the mucosal membranes, and in particular, the membranes of the eyes, mouth and nose. This typically occurs though contact of a mucosal membrane with a pathogen-covered body. This may, for example, occur when a young child puts a toy into their mouth. While such behaviour is usually only exhibited in young children, older children and adults can unwittingly allow pathogens to enter the body by touching the mouth, eyes or nose with hands that may be contaminated. A good hand washing routine is therefore a vital part of personal hygiene.

Whilst hand washing is an essential part of personal hygiene, it is not always possible and relatively few people will wash their hands in a manner sufficient to remove substantially all the pathogens. This is evidenced by the extra hand washing routines undertaken by healthcare professionals, and in particular, surgeons. Moreover, contamination of the hands can occur almost instantaneously by touching a contaminated article, such as a tap, door handle or towel.

It is estimated that the average person touches their face/head several hundred times a day, and the mouth or nose about four times per hour (Alonso et al., Clin. Infect. Dis. (2013) 56 (4): 617). This provides numerous opportunities for pathogens that may be present on the hands to enter the body via the mucosal membranes of the nose, mouth and eyes. Healthcare professionals consequently recommend avoiding touching the face to minimise the risk of contracting illnesses that may be spread by direct contact. Diseases spread by direct contact include the common cold, influenza, Ebola, polio, conjunctivitis, Legionella, meningitis, MRSA, plague and SARS (severe acute respiratory syndrome).

Diseases caused by direct contact can reach pandemic levels relatively quickly, as recently shown by the H1N1 “swine ‘flu” pandemic of winter 2009/2010. This is estimated to have caused about 17,000 deaths. However, even “normal” seasonal influenza is surprisingly deadly. The Center for Disease Control (CDC) estimates that in the US between winter 1976 and spring 2007, ‘flu-associated deaths ranged from a low of about 3,000 to a high of about 49,000 people per year.

It is worth noting that during ‘flu epidemics, healthcare professionals advise people to stop touching their face, see, for example, http://www.webmd.com/cold-and-flu/features/prevent-flu-dont-be-touchy. As a recent example of how dangerous touching your face can be, in October 2014 it was reported that a Spanish nurse may have picked up Ebola by touching her face after treating an Ebola patient (see http://www.bbc.co.uk/news/world-europe-29539444).

Infections can also be spread from other parts of the body to the face by touching the infected area and then the face. An example of an infection that can be spread in this way is herpes, caused by the herpes simplex virus. Warts, caused by human papillomavirus, can also be spread from the body to the face in this way.

In addition, existing infections can be spread around the face by touching. For example, infections caused by Propionibacterium acnes lead to the skin condition acne. Dermatologists treating a person with acne will often recommend that they reduce contact between the hands and the face to avoid both transferring any bacteria or dirt that may be on the hands to the face, and to avoid spreading the existing infection around the face.

Most people are unaware that they touch their face so often. As a subconscious or even reflex action it is difficult to control and many people, even if they are aware of the habit, will struggle to reduce the frequency of such facial contact.

Masks which cover the mouth and nose can be used as a barrier method to try and minimise the risk of contracting an infection. However, the efficacy of such masks in preventing infection is unclear. While they may help to prevent an infected person from spreading their illness via coughing/sneezing, they provide no barrier to pathogens entering the body via the eyes. Use of such masks can cause unnecessary alarm and panic amongst the population, and may lull the wearer into a false sense of security, leading them to take less care with other hygiene measures.

It is desirable to find ways of reducing the number of times a human subject will touch their face with their hands. It is also desirable to provide methods of changing the behaviour and/or habits of a human subject, such that after adhering to the method the human subject touches their face less frequently.

### SUMMARY

The present application seeks to address this need by making contact between the hand and the face of a subject an unpleasant experience. This will inevitably reduce the number of times the subject touches the face, and should therefore reduce the exposure of the face of the subject to infectious diseases.

Thus, in one aspect the present invention relates to a method of improving hygiene of a human subject by discouraging contact between the hand and the face of said subject. In particular, the method of the present invention discourages contact with mucosal membranes of the face of the subject.

In a first embodiment, the present application provides a method of improving the hygiene of a human subject, wherein the human subject wears an article covering at least part of the hand, said article being at least partially coated or impregnated with one or more irritants characterised in that said subject wears the article for at least 10% of the time in a one hour period.

In a second embodiment, the present application is directed towards a method of improving the hygiene of a human subject wherein said subject applies a composition comprising one or more irritants to at least part of their hand characterised in that said subject allows the irritant to remain on the at least part of the hand for at least 10% of the time in a one hour period.

In a third embodiment, the present invention is directed towards a hand covering article made from an absorbent material, wherein substantially the entire surface (e.g. the outer surface) of the hand covering article is coated or impregnated with one or more irritants.

Preferably, the irritant used in the first, second and third embodiments does not irritate the skin of the hand.

The skin on the human face is significantly more sensitive than the skin on the rest of the body. Without wishing to be bound by theory, it is thought that this increase in sensitivity is caused by thinner skin on the face, and possibly also by an increased number of pores. The reasons for this difference are unclear, but it is thought that thinner facial skin allows for more facial expression, which might otherwise be difficult to achieve with thicker skin. Thus, the irritant used in the first, second and third embodiments does not irritate (unbroken) skin of the hand, but does cause irritation to the skin and mucosal membranes of the face, particularly to areas of broken and/or inflamed skin.

Preferably, the irritant used in the first, second and third embodiments does not irritate unbroken skin. Typically, the irritant causes an unpleasant sensation in the human subject when contact occurs between the hand of said subject and a mucosal membrane of the face or region of skin on the face of said subject.

In a fourth embodiment, the present application is directed towards kits for use in the methods of the present invention.

The kit of the invention preferably comprises
(a) at least one hand covering article that is at least partially coated or impregnated with an irritant;
(b) a hand sanitizer composition; and
(c) optionally instructions for use in a method of the invention.

Methods of forming hand covering articles that are at least partially coated or impregnated with one or more irritants are also described.

These and other embodiments of the present invention are discussed in more detail below.

### DETAILED DESCRIPTION

While some of the contact between a person's hand and their face will be intentional (such as removing morsels of food/drink from the lips or pushing hair out of the eyes), most of the contact between a person's hand and their face is a subconscious action. For example, it is well known that people may scratch their head while thinking or rub their eyes while tired. In addition, some people have so-called “bad habits” that involve touching the face with the hands. Examples of such habits include nose-picking, thumb-sucking, skin or spot (acne) picking and nail-biting. Persuading people to abandon such bad habits is difficult. Oftentimes, it may be a way for that person to soothe themselves. Thumb-sucking is an example of this.

Generally speaking, face touching is unhygienic and can lead to the spread of diseases and infection. In scenarios where pandemic infection is a serious public concern (such as during a ‘flu pandemic), any measures which reduce the spread of infection should be encouraged. However, simply reminding or encouraging the general public to wash their hands and avoid touching their face is likely to have little impact on the spread of infection. Simply speaking, most people do not realise that they are touching their face so often, so even with improved awareness of the issue the actual rate of face touching is largely unchanged. It is therefore preferable to provide a method that changes the behaviour and/or habits of a person, such that these subconscious actions are reduced in frequency or even completely avoided.

Thus, in the context of the present invention, a “method of improving hygiene” can be viewed as a method of reducing the frequency that a human subject touches their own face, or a method of reducing unhygienic touching of their face by a human subject.

The present invention seeks to achieve this by making contact between the face and the hand an unpleasant sensation. The unpleasant sensation serves to make the person consciously aware of when they have touched their face. In this way, the person subconsciously touching their face will recognise those times when their habit has caused them to touch their face, and they will (over a period of time) learn to control their habit so as to avoid repeating the unpleasant experience.

In order to make contact between the face and the hand less desirable, in a first embodiment, at least one of the hands may be covered in an article that is at least partially coated or impregnated with one or more irritants. Said article may cover part or all of the hand, depending on the situation.

As used herein, a hand is defined as an extremity located at the end of a human subject's arm, usually including fingers and a thumb (collectively known as digits), a palm and a “heel” (the area where the palm meets the wrist).

As used herein, the terms “article”, “article for covering” and “hand covering article” are used essentially interchangeably, and all relate to the same article for covering at least part of the hand of a human subject. The hand covering article is used in the first embodiment of the invention. As the method of the invention can be viewed as a method for changing the behaviour of a human subject, the hand covering article can be viewed as a behaviour modifying device. By “behaviour modifying device” is meant a device that, when used, helps the user to modify their behaviour. As used herein, “behaviour modifying device” is entirely interchangeable and synonymous with “hand covering article”; the behaviour modifying device is the hand covering article as described herein. The behaviour modifying device therefore covers at least part of the hand.

The hand covering article covers the hand, so can be defined by at least two surfaces, an “inner” surface and an “outer” surface. The inner surface is the surface in contact with the skin of the user's hand when the hand covering article is being worn, and the outer surface is the opposite surface which would be brought into contact with anything touched by the user when wearing the hand covering article.

The article covering the hand may in one embodiment cover the whole hand including the fingers, the thumb, the palm, the back and the heel of the hand. The covering may also extend part way along the arm, covering the wrist and optionally part of the forearm.

It is not necessary for both hands to be fully or partially covered by the hand covering article, as most people will have a dominant hand and will use this hand to touch their face more often than the other, less dominant hand. For most people, the dominant hand is the right hand.

Within the context of the present application, the words “hand” and “hands” are used interchangeably, as the hand covering article (or, as described below, the irritant applied directly to the hand) may be applied to one or both of the hands.

If both hands are to be fully or partially covered by a hand covering article it is not necessary for the same hand covering article(s) to be used on each hand. For example, it may be desirable for the hand covering articles to cover the forefinger and thumb only on one hand, and the forefinger only of the opposite hand.

Preferably, the article for covering the hand comprises at least one sheath fora digit.

By “sheath for a digit” is meant a pocket of material that is proportioned to closely envelop a finger or thumb of the human subject.

Articles suitable for covering the whole of the hand may have only a single sheath for the entire hand, or separate sheaths for the fingers and the thumb. Preferably, the hand covering article may have separate sheaths for all four fingers and a separate sheath for the thumb.

In a preferred embodiment, the hand covering article for use in the present invention is a glove, i.e. a garment covering the whole hand, having separate sheaths for each finger and the thumb.

In another embodiment, the hand covering article for use in the present invention is a mitten, i.e. a garment covering the whole hand, having a single sheath for all of the fingers and a separate sheath for the thumb.

It is uncommon to touch the face with the back of the hand and/or the palm of the hand. Consequently, in some embodiments the hand covering article may have an open section for the back of the hand and/or an open palm. This can avoid the hand becoming too hot when carrying out the method of the invention for prolonged periods of time.

In order to retain maximum use of the hand whilst covered, the hand covering articles are preferably sized to fit a human hand.

Articles covering part of the hand may comprise individual sheaths for covering the separate fingers/the thumb. For example, a single sheath for the index finger may be desirable, as this is the finger most likely to be used to touch the face. A single sheath for the thumb may also be desirable, if a particular subject has a habit of thumb-sucking.

As it is generally only the tips of the fingers/thumb that contact the face, the sheaths do not need to extend all the way down the digit. For example, a sheath that only covers the tip of one or more digits is envisaged as a suitable hand covering article for use in the present invention.

The hand covering article may be a single sheath for an individual digit, or may comprise two or more sheaths for two or more digits. Typically, the index finger and thumb will be covered.

As an alternative, multiple such single sheath articles may be worn at any one time. For instance, the first embodiment may comprise using one or more “thimbles” (i.e. small sheathes that cover only the tips of the fingers/thumb) as the hand covering article.

Any configuration of finger and thumb sheaths with optional covering for the front and/or back of the hand is possible. For example, an article may comprise sheathes for the index finger, second finger and thumb and covering for the back of hand only. Alternative articles may comprise separate sheathes for each of the fingers and the thumb, and cover the back of the hand, but leave the palm of the hand uncovered. Still other alternative articles may comprise separate sheathes for each of the fingers and the thumb, and cover the front of the hand, but leave the back of the hand uncovered.

The hand covering articles may be either loose or held in place by a fastening means. Suitable fastening means include an elastic material, a buckle, a snap fastener, a tether or a Velcro® style “hook and loop system”.

Typically, the fastening means is at the opening of the sheath where the individual digit is inserted, and/or at the opening of the article where the entire hand is inserted. For an article covering the entire hand, the fastening means is typically positioned such that the article can be fastened around the wrist.

The article may have an elasticated portion at the wrist (e.g. an elasticated cuff), a buckle or a slit that may be closed with either a snap fastener or a Velcro® style “hook and loop system”.

For example, the article may have an elasticated region that is positioned in registration with the human subject's wrist while being worn.

For articles that do not cover the entire hand, the article may comprise tether connecting the one or more sheathes to the fastening means. Typically, two tethers could be used, respectively positioned to connect the fastening means to the sheath or sheathes over the front and the back of the hand of the human subject, such that when the fastening means is positioned at the wrist the sheathes are held on the one or more digits which they envelop.

Preferably, the hand covering articles have an elastic region. The elastic region can be located to fasten the sheath at the base of the digit, and/or to fasten the article to the wrist of the human subject.

In preferred embodiments, the entire article is elasticated. This ensures that the article is urged into conformity with the shape of the hand of the human subject during use. In such embodiments, the subject using the article is able to retain fine motor skills, reducing the impact of wearing the article on daily tasks and increasing the likelihood that the article will be worn for longer periods of time.

The hand covering articles for use in the present invention can be made of any material. It is, however, desirable that the wearer can perform their usual activities, and malleable materials are therefore preferred such that the wearer retains freedom of movement. Suitable materials therefore include leather, wool, silk, cotton, bamboo, flax, jute, nylon, polyester and acrylic. However, as the hand covering article is coated or impregnated with an irritant, the material the hand covering article is made from must be capable of being impregnated and/or coated by the irritants. Thus, in one embodiment, the hand covering articles are formed from an absorbent material (i.e. a material which is capable of absorbing a liquid composition, such as a nonwoven, a textile or a foam). In a preferred embodiment, the hand covering articles are made from a textile material. Suitable textiles include wool, silk, cotton, bamboo, flax, jute, nylon, polyester and acrylic.

In a preferred embodiment, the hand covering articles comprise cotton.

Alternative absorbent materials other than textiles include nonwoven fabrics, such as felt, spunbond webs, meltblown webs, spunlace webs, needlepunched webs and the like. Such materials often have lower tear strengths than textiles, and so a not always suitable for use in forming gloves and the like that are intended to be worn for prolonged periods. As such, textiles, i.e. fabrics formed from long fibres that are woven or knitted, are preferred.

Preferred materials include an elastic component, to ensure that the hand covering article has a snug fit and does not unduly limit manual dexterity of the hand. An example of such a material is a cotton/elastane blend.

The hand covering articles do not need to be made of a single material. It may, for example, be desirable to have sheathes made of one fabric, and coverings for the back and/or front of the hand in a different material. For example, the sheathes may be made of cotton or a cotton elastane blend, while the back and/or front of the hand covering may be made of a lighter, more breathable, material, such as gauze or Gortex®. This may be advantageous in warmer climates.

The mere fact that the wearer is using a hand covering article may be sufficient to discourage contact between the hand of the subject and the subject's face, as the sensation of contact will be different from touching the face with bare hands. Certain bad habits, such as nose and spot (acne) picking, will also be considerably harder to carry out whilst wearing a hand covering. In addition, if the hand covering articles are made of a light coloured material, dirt will be more visible than it might otherwise be. This alone would alert the wearer to the fact that their hands pick up a significant amount of dirt and debris even when carrying out normal tasks that would not be considered to be “dirty”, such as using a telephone or handling money. This would act as an incentive for the person to avoid contact with the face until the hands could be properly cleaned.

Nevertheless, it is preferable that at least part of the hand covering article is coated or impregnated with an irritant. The presence of an irritant impregnated on or covering the hand covering articles will make the experience of contact between the hand and face of that subject more unusual and unpleasant.

Even though the skin of the hands is generally thicker than the skin of the face, prolonged contact between the irritant and the skin of the hand may become uncomfortable, and therefore it is preferable that the irritant is not in direct contact with the skin of the hand and/or arm when the hand covering article is in use. Typically, this can be achieved by applying the irritant to the outer surface of the article, such that the inner surface (i.e. the surface intended to be in contact with the skin of the hand) does not have any irritant.

Suitable locations for the irritant include the sheathes for the digits and the part of the hand covering article located over the knuckles and the back of the hand. In some embodiments, the entire hand covering article is coated or impregnated with the irritant. The irritant preferably does not irritate unbroken skin.

In other words, the irritant does not cause any sensation when in contact with normal, undamaged skin. Likewise, it is preferred that prolonged exposure of the irritant to unbroken skin does not cause any adverse effects.

In some embodiments, substantially the entire surface of the hand covering article is coated or impregnated with the one or more irritants.

By “substantially the entire surface” is meant at least 75% of the surface is coated or impregnated with the one or more irritants, preferably at least 90% of the surface is coated or impregnated with the one or more irritants.

The irritant causes an unpleasant sensation when contact is made between the irritant and the face, particularly the mucosal membranes of the eyes, nose and mouth and any areas of skin that are already irritated or broken by infection. As the subject will obviously try to avoid this unpleasant sensation, the subject will adjust their behaviour, and will touch their face less and less.

By “unpleasant sensation” is meant an unusual sensory experience that is not desirable. Although such sensations are dependent on the individual human subjects carrying out the method of the invention, typically these would be described as tingling, stinging, warming, prickly or itchy sensations being experienced when the irritant is in contact with the face, preferably the mucosal membranes of the eyes, nose and mouth and any areas of skin that are already irritated or broken by infection.

The nature and amount of irritant is such that, in the event of contact with the face and particularly the mucosal membranes of the eyes, nose and mouth, medical treatment is not required to remove or mitigate the unpleasant sensation. The irritant does not therefore lead to any permanent state which must be alleviated by medical treatment by a physician or other specialist.

The irritants used in the present invention are preferably non-toxic and non-corrosive. It is further desirable that the irritants are not flammable.

The irritant should cause a temporary unpleasant sensation when it makes contact with the mucosal membranes of the eyes, nose and mouth and any areas of skin that are already irritated and/or broken by infection. The purpose of the unpleasant sensation is simply to make the human subject acutely aware that the face has been touched, ensuring that any subconscious face touching is noticed by the subject and any conscious face touching will be avoided.

The irritant is coated on or impregnated in the article covering the hand. Any irritation that may occur as a result of touching the face with the coated or impregnated article could in most circumstances be alleviated by simply moving the hand such that the article coated or impregnated with the irritant is no longer in contact with the face. However, in the event that some of the irritant has been transferred to the face, it may be necessary to remove the irritant from the skin of the face, for example by rinsing the area with water or with tears. The irritant may, for example, cause the eyes of the subject to water if the subject touches its eye with either an article that is coated or impregnated with said irritant, or with a part of the hand that is at least partially directly coated with said irritant. However, the unpleasant sensation caused by the irritant is typically quickly alleviated by crying or rinsing the area with water such that the uncomfortable sensation is quickly removed, typically in less than about one minute.

Suitable irritants for use in the present invention include chilli extract (such as capsaicin and capsaicinoids), menthol, camphor, natural or “essential” oils (such as tea tree and eucalyptus), a tannin, and onion extracts comprising syn-propanethial-S-oxide.

In some embodiments, the irritant comprises chilli extract, preferably capsaicin or a capsaicinoid compound.

Preferred capsaicinoid compounds are selected from dihydrocapsaicin, nordihydrocapsaicin, homodihydrocapsaicin, homocapsaicin and nonivamide.

In some embodiments, the irritant comprises a natural or essential oil. Preferably, the natural or essential oil is tea tree oil or eucalyptus oil.

In some embodiments, the irritant comprises menthol.

In some embodiments, the irritant comprises camphor.

In some embodiments, the irritant comprises onion extracts.

In some embodiments, use of multiple irritants may be desirable. Thus, one possible combination of irritants for use in the present invention includes both capsaicin or a capsaicinoid compound and eucalyptus.

Use of tea tree oil as the irritant is particularly preferred as it is both a mild irritant and has been found to exhibit an antimicrobial effect against certain pathogens.

The hand covering article may be coated or impregnated with the irritant by the manufacturer, or by the user. For example, the irritant may be contained in a wet wipe, rub or spray that can be applied while the subject is wearing the hand covering article.

In one embodiment, the present application is directed towards a method of manufacturing a hand covering article made from an absorbent material and which is at least partially coated or impregnated with one or more irritants, comprising the steps of

(a) forming a hand covering article from an absorbent material, and

(b) at least partially coating and/or impregnating the hand covering article with one or more irritants.

In a further embodiment, the present application is also directed to a method of manufacturing a hand covering article made from an absorbent material and which is at least partially coated or impregnated with one or more irritants, comprising the steps of

(a) at least partially coating and/or impregnating the absorbent material with one or more irritants, and

(b) forming the hand covering article from said coated/impregnated absorbent material.

As indicated above, the absorbent material may be coated or impregnated with one or more irritants prior to manufacture of the hand covering articles or afterwards. Any reasonable method can be used to coat and/or impregnate the absorbent material either before or after formation of the hand covering articles. Thus, the absorbent material prior to manufacture of the hand covering articles or the shaped hand covering articles may be dipped into a tank comprising one or more irritants, typically in solution, or topical application of the irritant may occur, for example by spraying with a suitable composition comprising one or more irritants.

Topical application to the absorbent material is preferred, as this allows the irritant to be preferentially located on one side of the absorbent material rather than the other. The absorbent material can then be formed into the hand covering article such that the surface on which the irritant is located forms the outer surface of the hand covering article, such that when in use the irritant is not located on the surface in contact with the skin of the user's hand.

Thus, within the context of the present invention, it is preferable for the irritant to be on the “outer” surface of the hand covering article, that is to say that the irritant should preferably reside mainly on the external portion of the hand covering article, so as to reduce contact between the irritant and the skin of the hand during use. Consequently, topical application of the irritant to the hand covering article is preferred.

Topical application of the irritant to either the absorbent material prior to manufacture of the hand covering articles or to the hand covering article itself may take place by spraying the material/articles with a suitable irritant composition, or by rubbing the surface of the absorbent material with an irritant composition such as a wax or rub.

Compositions for spraying onto the absorbent material or the shaped hand covering articles will generally comprise one or more irritants and at least one suitable carrier. Suitable carriers are typically solvents and may be selected from water, alcohols (methanol, ethanol etc.), ethers (diethyl ether, tetrahydrofuran), acetone, dimethylsulfoxide, dimethyl formamide, dimethylactamide etc. Of these solvents, water and alcohols are preferred, with water, ethanol, and mixtures thereof being particularly preferred.

As the irritants suitable for use with the present invention irritate the mucosal membranes of the face, forming an aerosol spray of the irritant can lead to potential irritation of users during manufacture of the articles. While suitable precautions can be taken in a factory, individuals applying the irritant to a suitable absorbent hand covering article (for example those individuals applying the irritant when using one of the kits of the invention) may wish to avoid applying the irritant using a spray. As such, topical application via a wax or rub is particularly preferred in some scenarios, for ease of manufacturing.

Topical waxes/rubs comprise one or more irritants together with a suitable carrier, which is generally inert and skin-compatible. Suitable carriers for such waxes/rubs include inert waxes such as paraffin wax, beeswax, candelilla wax, soy wax and carnuba wax and/or “butters” such as shea butter or cocoa butter.

Irritants for use in the present invention vary in potency. For this reason, the amount of irritant comprised in a topical wax or rub may be anywhere between 0.1% by weight to 99% by weight. While this may seem unduly broad, some irritants will affect some people more than others. A good example of this is onion extract; people who regularly chop onions can develop a degree of “resistance” to the irritants in onions, and for such people cutting onions is less likely to irritate the eyes. Conversely, some people are acutely sensitive to the irritant compounds found in onions.

Chilli extracts are however known to be relatively potent, and when these irritants are used in the irritant compositions of application to the absorbent material or shaped hand covering article, they tend to be used in relatively small amounts such as less than about 10% by weight, typically less than about 8% by weight or less than about 5 by weight %. Depending on the specific chilli extract to be used, it may only need to be present in very small amounts, such as 0.1%-5% by weight. It is noted that pepper sprays for self-defence or for use in law enforcement typically comprise a chilli extract in an amount of 0.1 to 3 wt. % and an alcohol solvent.

Capsaicin itself is a waxy solid, having a melting point of around 62-65° C. It is therefore particularly suited to forming into a wax/rub for application to hand covering articles made from an absorbent material. The irritant composition can be suitably formulated to ensure the proper consistency. In some embodiments, the consistency may be similar to a skin cream. In some embodiments, the consistency may be more wax-like, such that it is a block that can be rubbed over a surface to leave a residue (i.e. the irritant composition at room temperature has a similar consistency to that of butter at 5° C.).

Advantageously, the hand covering article may also be coated or impregnated with other compounds. Such additional compounds may reduce irritation of the hand whilst the hand covering articles are in use, and/or may have a desirable cosmetic effect. Suitable additional compounds include aloe vera extracts, witch hazel, shea butter, cocoa butter, and natural oils (coconut, avocado, jojoba, olive, almond, argan, vitamin E). Such additional compounds may be added to the absorbent material prior to manufacture of the hand covering articles or the shaped hand covering articles after manufacture, and may be applied as part of the irritant composition, or separately.

In addition to, or instead of, the additional compounds listed above, the hand covering articles may further comprise additives that can provide an antibacterial effect, such as silver, copper or copper alloys.

Thus, in one embodiment, the hand covering articles used in the present invention further comprise silver, copper or copper alloys. Such metals can be incorporated into the hand covering articles by weaving metal-impregnated fibres into the textile when manufacturing the hand covering articles, or by treating the hand covering articles with a composition comprising a colloidal solution of the above metals.

If the subject does not wish to wear a hand covering article or is otherwise prevented from wearing a hand covering article in accordance with the present invention (e.g. military personnel), the second embodiment of the method of the invention can be practiced by applying the irritant directly to the hand of the subject, for example by using a gel, foam, spray, cream or balm containing the irritant. Until such time that the subject washes the irritant off its hand, the presence of the irritant should reduce unnecessary contact between the hand and face of that subject, particularly the mucosal membranes of the face, and any areas of skin that are already irritated or broken by infection.

Thus, the present application further relates to a method of improving hygiene in a human subject characterised in that the subject applies a composition comprising one or more irritants to at least part of the hand such that the irritant is retained on the at least part of the hand.

Preferably, the at least part of the hand which retains the irritant does not contain any broken skin, and the irritant does not irritate unbroken skin.

Preferably, the method further comprises washing the at least part of the hand which retains the irritant to remove the irritant.

As with the hand covering articles discussed above, it is not necessary for the irritant to fully cover one or both of the hands, what is important is that the parts of the hand that are likely to come into contact with the face are coated. For most people, this will mean that at least the pad of the index finger should coated in the composition, as this is the part of the hand most likely to be used to touch the face. For individuals with a particular habit, such as thumb-sucking, coating the thumb and index finger only may be sufficient.

If both hands are to be covered by the irritant, the extent to which each hand is covered may vary from hand to hand, or may be the same. For example, it may desirable to apply the irritant to the thumb and index finger of one hand, and to the back of the other hand only.

Irritants for use in this embodiment of the invention will be same as those described above. Again, it is important that the irritant only produces an unpleasant sensation when in contact with the mucosal membranes of the eyes, nose and mouth and any areas of skin that are already irritated or broken by infection, as does not unduly irritate the skin of the hand.

Formulations for application of the irritant composition to the hand may take any form. Suitable formulations include powders, gels, oils, foams, spray (such as pump spray or aerosol spray), creams and balms.

In a preferred embodiment, the composition comprising the irritant is a hand sanitizer composition.

Hand sanitizer compositions are widely used in both domestic and healthcare settings. Hand sanitizer compositions can be applied to to kill pathogens, and are useful in situations where it is not possible to wash the hands with soap and water. The hand sanitizing compositions are typically applied to both hands, and the hands are rubbed together. The main components in the sanitizer composition will evaporate off, killing pathogens and generally leaving a thin film behind, which may provide a lasting antimicrobial effect. It is not necessary to wash off hand sanitizer compositions after use.

The most common type of hand sanitizer composition comprises an alcohol as active ingredient. For maximum efficacy, the WHO recommends that alcohol based hand sanitizers comprise 60-80 wt. % alcohol.

Suitable alcohols for use in hand sanitizer compositions can include any water-soluble alcohol known in the art. Non-limiting examples of suitable alcohols include methanol, ethanol, propanol, isopropyl alcohol, butanol, t-butanol, 2-butanol, pentanol, hexanol, or combinations thereof. Typically, the alcohol is ethanol, isopropyl alcohol or combinations thereof. More typically, the alcohol is ethanol or denatured alcohol.

Alcohol-free sanitizers are also known, and these use antimicrobial agents such as benzalkonium chloride, benzethonium chloride, methylbenzethonium chloride, hexylresorcinol, chlorhexidine gluconate, para-chloro-meta-xylenol, chloroxylenol, cloflucarban, fluorosalan, hexachlorophene, iodine complexes such as ammonium ether sulfate and polyoxyethylene sorbitan monolaurate or a phosphate ester of alkylaryloxy polyethylene glycol, iodine tincture, iodine topical solution, nonylphenoxypoly (ethyleneoxy)ethanolidine, poloaxmer-iodine complex, triple dye, povidone-iodine complex, undecoylium chloride iodine complex, mercufenol chloride, methylbenzethonium chloride, phenol, secondary amyltricresols, sodium oxychlorosene, tribromsalan, triclocarban, triclosan, calomel/oxyquinoline benzoate/triethanolamine/phenyl derivative combination, and mercufenol chloride/secondary amyltricresols as the active ingredient.

Combinations of these disinfectants may also be used. Preferably, the disinfectant is selected from the group consisting of chlorhexidine, chloroxylenol, hexachlorophene, iodine, iodophors, quaternium ammonium compounds such as benzalkonium chloride or benzethonium chloride, triclosan, and combinations thereof.

Hand sanitizers comprising both alcohol and at least one further antimicrobial agent as listed above are also known.

Hand sanitizer compositions typically comprise additional components beyond the active ingredient(s) for ease of use. Thus, hand sanitizer compositions may comprise agents to improve the viscosity of the sanitizer. Suitable viscosity enhancers include clays and derivatives thereof, silicates, silicas and derivatives thereof, and combinations thereof. Suitable clays and derivatives thereof include, but are not limited to, bentonite and derivatives thereof such as quaternium-18 bentonite, hectorite and derivatives thereof such as quaternium-18 hectorite, montmorillonite, and combinations thereof. Suitable silicates include, but are not limited to, magnesium aluminium silicate, sodium magnesium silicate, lithium magnesium silicate, tromethamine magnesium aluminium silicate, and combinations thereof. Suitable silicas and derivatives thereof include, but are not limited to, silica, hydrated silica, hydrophobic silica, silica silylate, silica methyl silylate, colloidal silicone dioxide, fumed silica, and combinations thereof. Other examples of suitable viscosity enhancers include polyolefin resins, lipophilic/oil thickeners, ethylene/vinyl acetate copolymers, polyethylene, cetyl hydroxy ethyl cellulose, hydroxyethylcellulose, hydroxypropyl methylcellulose, hydroxypropylcellulose, other organically modified celluloses, PVP/decane copolymer, PVM/MA decadiene crosspolymer, PVP/eicosene copolymer, PVP/hexadecane copolymer, butylated PVP, carbomers, acrylic based thickeners, polyethylene glycol 600, polyethylene glycols, myristyl alcohol, cetyl alcohol, stearyl alcohol, behenyl alcohol, PEG-150 distearate, PEG-175 diisostearate, polyglyceryl-10 behenate/eicosadioate, disteareth-100 IPDI, polyacrylamidomethylpropane sulfonic acid, silicone crosspolymers, polyamide blends, and combinations thereof.

Hand sanitizer compositions can also include various preservatives to increase the shelf life of the sanitizer. Some suitable preservatives include potassium sorbate, tetrasodium EDTA; iodopropynyl butylcarbamate, benzoic esters (parabens), such as methylparaben, propylparaben, butylparaben, ethylparaben, isopropylparaben, isobutylparaben, benzylparaben, sodium methylparaben, and sodium propylparaben, 2-bromo-2-nitropropane-1,3-diol, benzoic acid, amidazolidinyl urea and diazolidinyl urea.

The pH of a hand sanitizer should be approximately neutral (i.e. pH 7 or thereabouts). If necessary, pH modifiers may be added to the sanitizer composition to achieve the desired pH level. Any suitable acid or alkali material may be used as a pH modifier. For instance, some examples of basic pH modifiers that may be used in the sanitizer include, but are not limited to, ammonia; mono-, di-, and tri-alkyl amines; mono-, di-, and tri-alkanolamines; alkali metal and alkaline earth metal hydroxides; alkali metal and alkaline earth metal silicates; and mixtures thereof. Examples of acidic pH modifiers that may be used in the present disclosure include, but are not limited to, mineral acids; and carboxylic acids; and polymeric acids. Specific examples of suitable mineral acids are hydrochloric acid, nitric acid, phosphoric acid, and sulfuric acid. Specific examples of suitable carboxylic acids are citric acid, glycolic acid, lactic acid, maleic acid, malic acid, succinic acid, glutaric acid, benzoic acid, malonic acid, salicylic acid, gluconic acid, and mixtures thereof. Specific examples of suitable polymeric acids include straight-chain poly(acrylic) acid and its copolymers (e.g., maleic-acrylic, sulfonic-acrylic, and styrene-acrylic copolymers), cross-linked polyacrylic acids having a molecular weight of less than about 250,000, poly(methacrylic) acid, and naturally occurring polymeric acids such as carageenic acid, and alginic acid.

Hand sanitizer compositions can be drying to skin, particularly those that are alcohol based. It can therefore be desirable to incorporate emollients to provide a desirable moisturizing effect when the sanitizer is used. Suitable emollients include oils such as petrolatum based oils, petrolatum, vegetable based oils, mineral oils, natural or synthetic oils, lanolin and its derivatives, fatty esters, glycerol esters and derivatives, propylene glycol esters and derivatives, alkoxylated carboxylic acids, alkoxylated alcohols, fatty alcohols, fatty acids, and combinations thereof.

Incorporating an emollient into the hand sanitizer compositions has the further advantageous effect that the subject is less likely to feel the need to subsequently apply a topical emollient to obtain a moisturised feeling. This is beneficial within the context of the present invention, as touching a container in order to dispense e.g. hand lotion could lead to rapid re-contamination of the hand.

Hand sanitizer compositions can also comprise further agents that may be beneficial to skin, such as hydrolysed wheat protein, glycerine and glycerine derivatives, sodium hyaluronate, hyaluronic acid, betaine, amino acids, glycosaminoglycans, honey, sorbitol, glycols, sugars, polyols, hydrogenated starch hydrolysates, sodium PCA, lactic acids and lactates, urea and natural extracts such as aloe vera extract, witch hazel extract, chamomile extract, echinacea extract and lavender extract, allantoin, keratin, methyl glucose dioleate, shea butter, anti-aging agents, anti-inflammatory agents, skin lipids, enzymes, scar care agents, vitamins, minerals and sunscreens.

Hand sanitizer compositions may comprise additives to control the colour and/or fragrance of the resulting composition. Any suitable fragrance or colour may be used. However, “fresh” or neutral colours/scents are preferred. Suitable scents include citrus, ocean scents and floral scents.

The hand sanitizer compositions for use in the present invention further comprise at least one irritant, as described above. The at least one irritant may be added to the hand sanitizer composition before, during or after formulation.

In order to help protect the skin of the hand from the at least one irritant, it may be desirable to include a skin protectant in the hand sanitizer compositions. Skin protectants improve the barrier properties of the skin, and can reduce the skin's sensitivity to external irritants.

Silicones are a class of compounds that can act as skin protectants. Suitable silicone materials include, for example, a silicone surfactant, a volatile silicone oil, a non-volatile silicone oil, or combinations thereof. More particularly, the silicone material may be, for example, dimethicone, cyclomethicone, polyalkylsiloxanes, polyarylsiloxanes, polyalkylarylsiloxanes, polysiloxane gums, polyether siloxane copolymers, and combinations thereof. Exemplary silicone and silicone derivatives also include branched or linear cyclical silicone or silicone derivatives, cyclomethicone, dimethicone polysiloxane, dimethiconol, polysiloxanes, polysiloxane copolymers, polyalkyl aryl silanes, polyaryl siloxanes, polyalkyl siloxanes, polyalkyl aryl silanes, polysiloxane copolymers, alkyl dimethicones, alkyl methicones, alkyldimethicone copolyols, phenyl silicones, alkyl trimethylsilanes, cyclopentasiloxane, dimethicone crosspolymer, trisiloxane, and combinations thereof.

Preferably, the hand sanitizer compositions for use in the present invention are in gel or foam form, as these are easier to use than liquids. More preferably, the hand sanitizer compositions are formulated as a gel.

In a preferred embodiment, the hand sanitizer compositions for use in the present invention have alcohol as the main active ingredient. Preferably, the alcohol is present in an amount of at least 60 wt. %, for example at least about 70 wt. % or at least about 80 wt. %.

Preferably the alcohol used in the hand sanitizer compositions of the present invention is ethanol or denatured ethanol.

In an alternative embodiment, the irritant may be transferred to the hand using a wet wipe. Wet wipes consist of a sheet of fabric material that is impregnated with a cleansing composition. The wipes are rubbed over the surface to be cleaned and may be formulated with gentle cleansers for use on the body, or with more harsh cleansing compositions for use in cleaning hard surfaces.

Wet wipes for use on the body come in many forms. For example, it is possible to purchase wet wipes specifically for make-up removal and/or for cleansing the face, wet wipes for sanitizing wounds, wet wipes for babies and wet wipes for cleansing the hands. Such wipes are useful in situations where soap and water are not available. A wet wipe can be removed from a sealed packet, and wiped over the desired area of the body. The compositions that are on the wet wipe are typically fast drying such that it is not necessary to dry the body after use. Thus, wet wipes generally leave behind a thin film of residue after use.

The nature of the fabric material is not particularly important, but it must be capable of being impregnated with the cleansing composition, and should also be malleable, such that it can be wiped all over the hands. The fabric material of wet wipes typically comprises nonwoven fibrous sheet materials which include melt blown, coform, air-laid, bonded-carded web materials, hydroentangled materials and combinations thereof. Such materials can comprise synthetic or natural fibers or combinations thereof. Typically, the wet wipes define a basis weight of from about 25 to about 120 grams per square meter and desirably from about 40 to about 90 grams per square meter. Wet wipes may comprise a coform basesheet of polymeric microfibers and cellulosic fibers having a basis weight of from about 60 to about 80 grams per square meter and desirably about 75 grams per square meter. Methods of manufacturing such coform basesheets are described in U.S. Pat. No. 4,100,324. Such coform basesheets generally comprise a gas-formed matrix of thermoplastic polymeric meltblown microfibers, such as, for example, polypropylene microfibers, and cellulosic fibers, such as, for example, wood pulp fibers.

The relative percentages of the polymeric microfibers and cellulosic fibers in the coform basesheet can vary over a wide range depending on the desired characteristics of the wet wipes. For example, the coform basesheet may comprise from about 20 to about 100 weight percent, desirably from about 20 to about 60 weight percent, and more desirably from about 30 to about 40 weight percent of polymeric microfibers based on the dry weight of the coform basesheet being used to provide the wet wipes.

Alternatively, wet wipes can comprise a composite which includes multiple layers of materials. For example, wet wipes may include a three layer composite which includes an elastomeric film or meltblown layer between two coform layers as described above. In such a configuration, the coform layers may define a basis weight of from about 15 to about 30 grams per square meter and the elastomeric layer may include a film material such as a polyethylene metallocene film.

Individual wet wipes are generally arranged in a folded configuration. Such folded configurations are well known to those skilled in the art and include c-folded, z-folded, quarter-folded configurations and the like. Each wet wipe may also be interfolded with the wet wipes immediately above and below in the stack of wet wipes.

Wet wipes for use in cleansing the body are generally sized appropriately for the body part. For example, wet wipes for the specific removal of eye make-up may only be the approximate size of the eye socket, e.g. approximately 5×5 cm. Wet wipes for use in cleansing the hands are typically larger, and will generally have an unfolded length of from about 8 to about 30 cm and an unfolded width of from about 8 to about 30 cm.

The amount of cleansing composition contained within each wet wipe may vary depending upon the type of material being used to provide the wet wipe, the type of solution being used and the type of container being used to store the wet wipes. Generally, each wet wipe can contain from about 150 to about 600 weight percent and desirably from about 250 to about 450 weight percent solution based on the dry weight of the wipe. If the amount of solution is less than the above-identified range, the wet wipe may be too dry and may not adequately perform. If the amount of solution is greater than the above-identified range, the wet wipe may be oversaturated and soggy and the solution may pool in the bottom of the container.

Wet wipes may be individually packaged, or there may be several wet wipes packed in a single container. Where multiple wet wipes are packed in a single container, it is essential that the container is resalable to prevent the wipes from drying out.

The cleansing composition may simply comprise water. However, the cleansing compositions incorporated into wet wipes typically include a number of ingredients intended to enhance or impart particular properties to the wipe. Such properties may relate to cleaning efficacy, fragrance, medication, reduced irritation, skin health, aesthetics of the product and the like. Suitable ingredients used to provide such properties include emollients, surfactants, preservatives, chelating agents, pH buffers or combinations thereof.

Suitable surfactants can include sodium mono lauryl phosphate, sodium dilauryl phosphate, potassium mono lauryl phosphate, potassium dilauryl phosphate, diethanolamine mono lauryl phosphate, diethanolamine dilauryl phosphate, triethanolamine mono lauryl phosphate, triethanolamine dilauryl phosphate, sodium mono coco phosphate, sodium dicoco phosphate, potassium mono coco phosphate, potassium dicoco phosphate, triethanolamine mono coco phosphate, triethanolamine dicoco phosphate, sodium mono capric phosphate, sodium dicapric phosphate, potassium mono capric phosphate, potassium dicapric phosphate, triethanolamine mono capric phosphate, triethanolamine dicapric phosphate, and the like and combinations thereof.

The cleansing compositions generally have a pH of approximately 7 (i.e. neutral pH) to avoid skin irritation. In order to maintain this desirable pH, use of a pH adjusting agent may be required, such as citric acid or sodium hydroxide.

The cleansing compositions may include an effective amount of preservative to inhibit the growth of microorganisms. Suitable preservatives are well known to those skilled in the art and may include, for example, parabens, sodiumhydroxymethylglycinate, organic acids such as benzoic and malic acid, DMDM hydantoin and the like and combinations thereof.

As with the hand sanitizer compositions described above, cleansing compositions for wet wipes may comprise fragrance to provide a “fresh” or “clean” scent, such as citrus, floral or ocean scents.

Cleansing compositions may also comprise additives having beneficial properties, such as emollients to provide a moisturising effect. Emollients include oils such as petrolatum based oils, petrolatum, vegetable based oils, mineral oils, natural or synthetic oils, lanolin and its derivatives, fatty esters, glycerol esters and derivatives, propylene glycol esters and derivatives, alkoxylated carboxylic acids, alkoxylated alcohols, fatty alcohols, fatty acids, and combinations thereof.

As indicated above in relation to hand sanitizer compositions, incorporating an emollient into the wet wipes has the further advantageous effect that the subject is less likely to feel the need to subsequently apply a topical emollient to obtain a moisturised feeling. This is beneficial within the context of the present invention as touching a container in order to dispense e.g. hand lotion could lead to rapid re-contamination of the hand.

Cleansing compositions for wet wipes can also comprise further agents that may be beneficial to skin, such as hydrolysed wheat protein, glycerine and glycerine derivatives, sodium hyaluronate, hyaluronic acid, betaine, amino acids, glycosaminoglycans, honey, sorbitol, glycols, sugars, polyols, hydrogenated starch hydrolysates, sodium PCA, lactic acids and lactates, urea, natural extracts such as aloe vera extract, witch hazel extract, chamomile extract, echinacea extract and lavender extract, allantoin, keratin, methyl glucose dioleate, shea butter, anti-aging agents, anti-inflammatory agents, skin lipids, enzymes, scar care agents, vitamins, minerals and sunscreens.

As indicated above, the wet wipes may be used in the present invention to transfer an irritant to the hand. When the wet wipes additionally comprise an irritant, this will inevitably result in a thin film of irritant being left on the hand after use of the wet wipe. As with the other embodiments of the invention described above, the irritant is such that it causes only a temporary unpleasant sensation when contact is made with the skin of the face, and the mucosal membranes in particular.

The irritant will typically be added to the cleansing composition during manufacture of the wet wipe.

Wet wipes have an advantage over hand sanitizer compositions in that the wipes can also remove visible dirt from the hands. However, when wet wipes are used, the user will subsequently need to dispose of any used wet wipes.

The composition comprising one or more irritants (e.g. the hand sanitizing composition or wet wipe) may further comprise a UV dye. In such embodiments, the human subject can easily determine which parts of the hand have been covered with the irritant using a UV lamp, and furthermore whether the irritant has been removed after subsequent hand washing.

The methods described above may be used continuously until the subject's behaviour is changed, or may be used for short periods and repeated as necessary until involuntary or subconscious face touching has been eradicated or significantly reduced in that subject.

Clearly, how long the methods need to be carried out for will vary significantly from person to person. A single use of any of the methods for a relatively short period of time, e.g. 1 hour, may in some cases be adequate to alter behaviour, as certain subjects may only require being alerted to the fact that they touch their face so often to alter their general behaviour. Other subjects, particularly those with particular habits, such as nail biting, may require the method of the invention to be repeated periodically before their behaviour is altered such that they no longer perform that habit and subconsciously touch their face. Some subjects may be required to practice the method while sleeping.

Typically, the method is repeated, such that a plurality of short periods are used, as and when the subject finds the method convenient and/or appropriate. For example, daily use may be required. Once touching the face is no longer a habit, the methods of the invention may be discontinued, but the learned behaviour should remain.

Thus, in one embodiment, the invention relates to a method of improving hygiene of a human subject characterised in that said subject wears a hand covering article that is coated or impregnated with an irritant for at least 10% of the time in a one hour period, characterised in that the subject wears the article for at least one continuous period of at least five minutes.

In another embodiment, the invention relates to a method of improving hygiene in a human subject characterised in that said subject applies a composition comprising one or more irritants to at least part of the hand and leaves the composition on said hand for at least 10% of the time in a one hour period, characterised in that the subject leaves the composition on said hand for at least one continuous period of at least five minutes.

In order to obtain maximum effect, it is desirable that the methods are carried out continuously i.e. the hand covering article coated or impregnated with an irritant is worn, or the irritant remains on the hand, for a prolonged period of time. However, it is acknowledged that during any given time period, users will need to remove the hand covering articles to perform certain tasks that may be difficult to do when wearing the hand covering article, or may be impractical to do when the hand is coated with an irritant. For example, it may be impractical to prepare food while one or both of the hands are fully or partially coated with the irritant, as the irritant may spoil the food. Similarly, it would be necessary to wash the irritant from at least the fingers or remove hand covering articles that are coated or impregnated with an irritant before inserting or removing contact lenses. For this reason, in some embodiments, when the method of the invention is practiced for a waking day (approximately 16 hours), the hand covering article may only be worn, or the irritant left on the hand, for approximately 30% of that time.

Preferably, the method of the invention involves wearing the article (or allowing the irritant to remain on the skin of the hand) for at least 20% of the time in a 1 hour period, more preferably at least 30% of the time, more preferably at least 50% of the time in that period.

In such methods, the total period is preferably 2 hours, more preferably 4 hours, more preferably 8 hours, more preferably 12 hours, more preferably 16 hours and even more preferably 24 hours.

The total period may be a day or more, several days, a week, 10 days, two weeks, or a month or even longer, depending on how long it takes for the subject to alter their behaviour.

As the methods described herein are incompatible with certain daily tasks (such as inserting/removing contact lenses), this is unlikely to be a continuous period of e.g. 8 hour use, but is more likely to be an average period of 30 minutes per hour of the day.

For example, if a subject was to use any of the methods of the invention on a given day, they may not wish to wear any hand covering article or composition directly on the hand whilst dressing and eating, but may choose to do so whilst travelling or while in public places. Similarly, the subject may choose to wear the hand covering articles or have the irritant directly on the hand when performing tasks such as reading, writing, typing, drinking, using the telephone/other handheld technology and handling money. However, it is likely that the subject would not wish to wear any hand covering article or composition directly on the hand whilst washing, dressing, applying cosmetics, preparing food, eating or using the toilet. Thus, the subject may remove and reapply the hand covering article or irritant several times a day. Obviously, the irritant will be washed off the hand by normal routine hand washing and will need to be reapplied after each hand wash if the method of the invention is to be maintained.

Thus, the human subject preferably wears the article or allows the irritant to remain on the skin of the hand for at least one continuous period of at least 5 minutes, more preferably at least 10 minutes, more preferably at least 20 minutes, more preferably at least 30 minutes, most preferably at least 1 hour.

In embodiments wherein the total period is longer than an hour, it is preferable that the subject wears the article or allows the irritant to remain on the skin of the hand for at least one continuous period of at least 2 hours, preferably at least 3 hours. The advantage of leaving the irritant on at least part of one or both of the hands for such long periods is that the subject becomes less aware that the irritant is even there, making it more likely that the subconscious face touching will happen. This ensures that the irritant is present to make the subject acutely aware that subconscious face touching has taken place, providing the motivation for the subject to change their behaviour and reduce subconscious face touching in the future.

Viewed in another way, the present invention provides a method of improving hygiene of a human subject wherein, during a period of 1 hour, the human subject wears an article covering at least part of the hand that is at least partially coated or impregnated with one or more irritants or allows an irritant to remain on at least part of one of the hands for at least 10% of the time, more preferably at least 30% of the time, more preferably at least 50% of the time in that period.

In such methods, the total period is preferably 2 hours, more preferably 4 hours, more preferably 8 hours, more preferably 12 hours, more preferably 16 hours and even more preferably 24 hours.

The total period may be a day or more, several days, a week, 10 days, two weeks, or a month or even longer, depending on how long it takes for the subject to alter their behaviour.

In such methods, the human subject preferably wears the article for at least one continuous period of at least 5 minutes, more preferably at least 10 minutes, more preferably at least 20 minutes, more preferably at least 30 minutes, most preferably at least 1 hour, or in longer embodiments preferably at least 2 hours, most preferably at least 3 hours.

Viewed in another way, the present invention provides a method of improving hygiene of a human subject wherein, during a total period of 1 hour, the human subject:

(a) applies a composition comprising one or more irritants to at least part of their hand;

(b) allows said irritant to remain on the skin of their hand for at least one continuous period of at least 5 minutes; and

(c) washes their hand to remove the irritant,

wherein steps (a), (b) and (c) are repeated such that the irritant is allowed to remain on the skin of the hand of the human subject for at least 10% of the 1 hour total period, more preferably at least 30% of the total period, more preferably at least 50% of the total period.

Typically the irritant causes an unpleasant sensation in the human subject when contact occurs between the hand of said subject and a mucosal membrane of the face or region of skin on the face of said subject.

In such methods, the total period is preferably 2 hours, more preferably 4 hours, more preferably 8 hours, more preferably 12 hours, more preferably 16 hours and even more preferably 24 hours.

The total period may be a day or more, several days, a week, 10 days, two weeks, or a month or even longer, depending on how long it takes for the subject to alter their behaviour.

In such methods, the human subject preferably allows the irritant to remain on the skin of their hand for at least one continuous period of at least 10 minutes, more preferably at least 20 minutes, more preferably at least 30 minutes, most preferably at least 1 hour, or in longer embodiments preferably at least 2 hours, most preferably at least 3 hours.

The methods of the present invention may also be carried out while the subject is sleeping. The uncomfortable sensation caused by the irritant would likely wake the subject, alerting them to their behaviour. However, as the uncomfortable sensation is temporary, the subject should be able to return to sleep relatively quickly. Use of the methods of the invention overnight may be advantageous if a subject has a habit of thumb sucking in the night.

The methods of the invention may be used interchangeably, depending on the user and their personal situation. For example, use of the irritant directly on the hand may be preferable for some people, as this is more discreet than use of the hand covering articles. However, certain people may wish it to be known that they are taking active measures to improve their hygiene, and may therefore choose the hand covering articles over the direct composition.

As indicated above, the methods of the invention are incompatible with certain routine daily tasks. For example, the methods could not be practised while washing or performing tasks which involve getting one or both of the hands wet, as this would cause the irritant to be washed off the hand covering articles or the hand itself. The methods are also incompatible with performing tasks that deliberately involve contact with the face, and the mucosal membranes of the face in particular, such as inserting/removing contact lenses, inserting/removing facial piercings and applying cosmetics. Similarly, the methods would not be practical when going to the toilet or changing a diaper, or when applying/removing feminine hygiene products.

As the irritants for use in the present invention may irritate broken skin, the methods of the invention should preferably not be carried out while cleaning and/or dressing wounds.

The methods of the invention would also not be carried out by persons carrying out work that typically involves wearing gloves, such as horticultural/agricultural work, building work, medical work and working in a laboratory.

The methods of the invention are primarily intended for use during times when the hands are predominantly idle, or are involved in tasks which would not be considered to involve getting the hands “wet” or “dirty”. It is during these times, often when people are relaxing, that people are most likely to subconsciously touch their face, as they perceive their hands to be clean.

When people are carrying out obviously “dirty” and/or “wet” task (such as cleaning a toilet) they have a general awareness that their hands are unhygienic and that they therefore should not touch their face or other surfaces to avoid spreading germs. A majority of people would immediately wash their hands after performing such a task. In contrast, when people perceive that they are carrying out a “clean” task, they are much less likely to take hygiene precautions.

The crux of the present invention is to stop or significantly reduce subconscious face touching that occurs when the hands are perceived to be clean. While hands may well be “clean” in the sense that there is no visible dirt, they are unlikely to be sanitized, and can therefore still transfer pathogens to the face (or from the face potentially allowing the subject to spread its pathogens elsewhere).

Thus, in one embodiment the methods of the invention may be practiced whilst watching television, typing, using computers, using tablet computers, using telephones, working as a cashier, driving, visiting public spaces such as museums/galleries, cinemas/theatres, shops, gyms, restaurants etc., using public transport, carrying out needle- or paper-craft, using a shopping trolley or basket, handling money, working in an office, playing a musical instrument, reading, writing, walking, running, cycling and sleeping.

None of the tasks listed above would generally be considered to be “dirty tasks”, and most people would be unlikely to wash their hands after e.g. working in an office. However, it is often quoted in the media that the average office workstation, typically comprising a desk, computer and telephone, can have more germs on its surface than a toilet seat. Moreover, certain pathogens such as the cold and ‘flu viruses can survive for up to three days on hard surfaces. Consequently, carrying out so-called “clean” tasks can transfer significant pathogens to the hands. These pathogens will inevitably be transferred to the face once contact is made between the hands and the face.

In addition to methods of improving hygiene, the above method steps may be viewed as:

a method for changing the behaviour of a human subject, preferably wherein the behaviour is changed to reduce the frequency that the human subject involuntarily (or subconsciously) touches its face with its hand; or

a method of reducing the spread of infection in a human population, preferably wherein the infection is selected from common cold, influenza, Ebola, polio, conjunctivitis, Legionella, meningitis, MRSA, plague and SARS (severe acute respiratory syndrome);

wherein at least one human subject in said population practices the method(s) of improving hygiene as described above.

In a further embodiment of the invention, there is provided a kit comprising
(a) at least one hand covering article that is coated or impregnated with an irritant;
(b) a hand sanitizer composition; and
(c) optionally instructions for use in a method of the invention (preferably the method of the first embodiment).

In a further embodiment of the invention, there is provided a kit comprising
(a) at least one hand covering article;
(b) an irritant for application to the hand covering article;
(c) a hand sanitizer; and
(d) optionally instructions for use in a method of the invention.
   Component (b) is typically in the form of an irritant composition as described above.

In a further embodiment of the invention, there is provided a kit comprising
(a) an irritant for application to the hand;
(b) hand washing equipment; and
(c) optionally instructions for use in a method of the invention (preferably a method of the second embodiment).

Preferably, the irritant composition is a hand sanitizer composition or the at least one irritant is impregnated in a wet wipe as described above.

The irritant composition may also take the form of a wax or rub, as described above.

The irritant composition may also take the form of a sprayable solution, as described above, in which case the kit further comprises a pump spray for application of the irritant composition.

Use of a hand sanitizer composition or a wet wipe to transfer the irritant to the surface of the skin or hand covering article may be desirable if a large area is to be covered. Because of the more solid formulation, use of a wax or rub may be desirable if the irritant is only to be applied to a small area of the hand or hand covering article. This may be desirable if the subject has a particular habit of e.g. thumb sucking, and only application to the thumb area is required.

Kits may advantageously comprise multiple irritant compositions of different strengths and/or modes of application, such as a spray and a wax/rub. Thus, the spray could be applied to most, if not all of the hand covering article in an easy manner, and the wax could additionally be applied to areas most likely to make contact with the face, such as the pad of the forefinger. The user could then adapt the and covering articles to their own particular needs. A mix of lower and higher strength irritant compositions may also be desirable where the kits are for use within mixed age groups, as children are generally more sensitive to irritants than adults.

Preferably, the irritant composition or wet wipe further comprises a UV dye, and the kit optionally further comprises a UV light source.

Preferably, the kits of the invention also include an impermeable glove, preferably a glove made from nitrile, latex, vinyl, rubber or plastic, more preferably nitrile, latex or vinyl. Such impermeable gloves can be used to cover the hands temporarily allowing the subject to carry out a task that is not practical or inconvenient to do when wearing the hand covering article or when irritant is on the skin of the hands, such as using the toilet or eating. In this way, the hand covering article does not need to be continuously removed and applied, and in particular the composition on the hand does not need to be washed off and reapplied, every time the subject needs to carry out essential tasks through the day.

Kits of the invention may provide the user with numerous hand covering articles that can be used in the method of the invention, preferably in a disposable fashion. Thus the kits of the invention preferably comprise at least 10 hand covering articles. However, the kits of the invention may comprise any number of hand covering articles, from 10 to 1,000, e.g. about 20, about 50, about 100, about 200, about 500 hand covering articles. Smaller kits may be useful for individuals and small families, while larger kits can be useful in municipal settings, such as in schools, hospitals, airports etc.

Kits may comprise hand covering articles that are already coated or impregnated with an irritant. Alternatively, the kits may comprise an irritant (e.g. an irritant composition as described above) for application to the hand covering article.

The user wears the hand covering article for a period of time and then discards the article. The kit provides a hand sanitizer composition that the user can then apply to disinfect the hands after removal of the hand covering articles. The hand sanitizer composition may be a simple disinfectant composition, such as an alcohol based gel, or may optionally further comprise an irritant.

Hand sanitizer compositions are most effective on hands that have no visible dirt. For this reason, the kits advantageously further comprise hand washing equipment. The hand washing equipment comprises soap and disposable articles for drying the hands, such as paper towels or tissues. It is important that the articles for drying the hands are disposable, to minimise cross contamination either between uses or when the methods are carried out with multiple users.

Soaps may be in solid, liquid or foam form, and may comprise an antibacterial agent. Soaps may further comprise abrasive components to provide a physical scrubbing action. Suitable abrasive components include sugar, salt, ash, sand, clay, oatmeal, plastic micro beads and ground nut shells or kernels.

The hand washing equipment may further comprise water sterilization equipment, to prepare clean water for washing the hands.

The hand washing equipment may further include nail brushes to physically scrub dirt from the areas around and underneath the nails.

Kits may further comprise instructions for properly washing the hands and applying hand sanitizer compositions. While everyone thinks they know how to wash their hands, few people do it to a standard that would properly sanitize all surfaces and crevices of the hands. In fact, studies conducted amongst health care professionals (who are taught and regularly reminded to properly wash their hands) have found that on average only 72% actually do so (Szilagyi et al., BMC Infectious Diseases, 2013, 13:249).

The WHO provides guidelines on the proper washing of hands, see for example http://www.who.int/gpsc/tools/GPSC-HandRub-Wash.pdf. Typical hand washing steps are as follows: apply an amount of sanitizer or soap/water to the hands, and then (a) rub the palms of the hands together; (b) rub the right palm over the back of the left hand with fingers interlaced and vice versa; (c) rub the palms together with the fingers interlaced; (d) cup the hands together with finger tips overlapping to rub the finger tips; and finally (e) cup the thumbs and rub in a circular motion. If the hands are being washed with soap and water, the soap should be rinsed off, preferably under running water. The hands should be dried with a disposable towel, if available, and it is recommended that the towel is then used to turn off the tap, rather than touching the tap with clean hands.

The kits of the invention are intended for deployment in a variety of settings, including throughout the developing world where the spread of infectious diseases is often prevalent due to poor hygiene. In many situations, the language skills of the users may be unknown, and users may even be completely illiterate. Consequently, the instructions included in the kit will typically involve pictures demonstrating how the kit is to be used, and how the method of the invention can be practiced.

In view of the differing educational backgrounds of the potential users, and to avoid any inadvertent misuse of the hand covering article of the invention, it is preferred that the hand covering article contains a symbol which indicates that it is coated or impregnated with an irritant. For instance, a suitable symbol might be a picture of a chilli pepper, particularly when the irritant is capsaicin or a capsicinoid.

If present, the symbol is preferably in a prominent location, such as located on the outer wrist of the user when in use. Optionally, the symbol can be repeated to form a pattern across the entire surface of the hand covering article.

The use of a symbol marking the location of the irritant is advantageous if the kits contain both hand covering articles that have an irritant coated/impregnated thereon, and those that do not. This way, the user can easily determine which hand covering articles are coated or impregnated with the irritant.

Kits may additionally comprise a UV light and a suitable UV active composition which will brightly illuminate areas cleaned to acceptable standards, and show darker areas that are less well cleaned or vice versa. It is not anticipated that any user would use a UV light each time they washed their hands, but such UV lights can be useful educational tools, and may therefore be particularly suitable for addition to larger kits intended for schools, hospitals, airports etc. The UV light may also show where the composition comprising the irritant has been applied to the hand, and whether the irritant has been fully washed off, in embodiments where the composition comprising the irritant further comprises a UV dye.

The invention also provides the use of an article for covering at least part of the hand of a human subject to improve the hygiene of a human subject by reducing the frequency that the human subject involuntarily (or subconsciously) touches its face with its hand, characterised in that the article is at least partially coated or impregnated with one or more irritants.

The invention also provides a composition comprising one or more irritants for use in improving the hygiene of a human subject, by reducing the frequency that the human subject involuntarily (or subconsciously) touches its face with its hand, characterised in that the composition is applied to at least a part of the subject's hand.

The invention further provides a hand sanitizer composition comprising one or more irritants for use in improving the hygiene of a human subject, by reducing the frequency that the human subject involuntarily (or subconsciously) touches its face with its hand, characterised in that the composition is applied to at least a part of the subject's hand.

The invention further provides a wet wipe for use on the hand that is impregnated with a composition comprising one or more irritants for use in improving the hygiene of a human subject, by reducing the frequency that the human subject involuntarily (or subconsciously) touches its face with its hand, characterised in that the composition is applied to at least a part of the subject's hand.

The invention also provides the use of an article for covering at least part of the hand of a human subject to change the behaviour of said subject, e.g. by reducing the frequency that said subject involuntarily (or subconsciously) touches its face with its hand, characterised in that said article is at least partially coated or impregnated with one or more irritants.

The invention also provides the use of a composition comprising one or more irritants to change the behaviour of a human subject, by reducing the frequency that the human subject involuntarily (or subconsciously) touches its face with its hand.

The invention also provides the use of an article for covering at least part of the hand of a human subject to reduce the spread of infection in a human population, wherein the article is at least partially coated or impregnated with one or more irritants.

The invention also provides the use of a composition comprising one or more irritants to reduce the spread of infection in a human population, by reducing the frequency that at least some of the humans in said population will involuntarily (or subconsciously) touch their face with their hand(s).

The invention also provides the use of a hand covering article that is at least partially coated or impregnated with one or more irritants to reduce the spread of infection in a human population, by reducing the frequency that at least some of the humans in said population will involuntarily (or subconsciously) touch their face with their hand(s).

The invention also provides the use of a composition comprising one or more irritants to reduce the likelihood of a human subject contracting a disease spread by direct contact, by reducing the frequency that said subject will involuntarily (or subconsciously) touch their face with their hand(s).

The invention also provides the use of a hand covering article that is at least partially coated or impregnated with one or more irritants to reduce the likelihood of a human subject contracting a disease spread by direct contact, by reducing the frequency that said subject will involuntarily (or subconsciously) touch their face with their hand(s).

Diseases spread by direct contact include, but are not limited to, the common cold, influenza, Ebola, polio, conjunctivitis, Legionella, meningitis, MRSA, plague and SARS.

Preferred features of the invention may be combined in any manner. Thus certain features which are, for clarity, described herein in the context of separate embodiments, may be combined in any manner. Conversely, various features that are, for brevity, described in the context of a single preferred feature, may also be provided separately or in any sub combination.

1. A hand covering article made from an absorbent material, wherein substantially the entire outer surface of the hand covering article is coated or impregnated with one or more irritants, wherein said one or more irritants comprises chilli extract.

2. The hand covering article of claim 1, wherein at least 90% of the surface of the hand covering article is coated or impregnated with the chilli extract.

3. The hand covering article of claim 1, wherein the entire surface of the hand covering article is coated or impregnated with the chilli extract.

4. The hand covering article of claim 1, wherein the inner surface of the hand covering article does not have any irritant.

5. The hand covering article of claim 1, wherein the article is a glove.

6. The hand covering article of claim 1, wherein the chilli extract comprises capsaicin and/or capsaicinoids.

7. A method of changing the behaviour of a human subject, to reduce the frequency that the human subject involuntarily touches its face with its hand,
wherein the human subject wears an article covering at least part of the hand, said hand covering article being made from an absorbent material and wherein at least part of the surface of the hand covering article is coated or impregnated with chilli extract, and wherein said subject wears the article for at least 50% of the time in an eight hour period.

8. The method of claim 7, wherein the total period is at least 16 hours.

9. The method of claim 8, wherein the total period is at least one week.

10. The method of claim 7, wherein the subject wears the article for at least one continuous period of at least 2 hours.

11. The method of claim 7, wherein the hand covering article is as defined in claim 1.

12. Method of manufacturing a hand covering article as defined in claim 1, comprising the steps of
(a) forming a hand covering article from an absorbent material, and
(b) coating and/or impregnating substantially the entire surface of the hand covering article with chilli extract.

13. Method of manufacturing a hand covering article as defined in claim 1, comprising the steps of
(a) coating and/or impregnating the absorbent material with chilli extract, and
(b) forming the hand covering article from said coated or impregnated absorbent material.

14. A kit comprising:
(a) at least one hand covering article made from an absorbent material, wherein at least part of the surface of the hand covering article is coated or impregnated with chilli extract;
(b) a hand sanitizer composition; and
(c) optionally instructions for use of the article.

15. A kit comprising:
(a) at least one hand covering article;
(b) a composition comprising chilli extract for application to the hand covering article;
(c) a hand sanitizer composition; and
(d) optionally instructions for use of the article.

16. The kit of claim 14, wherein the chilli extract comprises capsaicin and/or capsaicinoids.

17. The kit of claim 14, wherein the chilli extract composition is formulated as either a topical spray or as a wax/rub.

18.-20. (canceled)
